(19) United States
(12) Patent Application Publication
(10) Pub. No.: US 2015/0297382 A1
(43) Pub. Date: Oct. 22, 2015

(54) Apparatus For Reducing Deployment Forces In A Self-Expanding Stent Delivery System

(71) Applicant: Boston Scientific Scimed Inc., Maple Grove, MN (US)

(72) Inventors: Anthony Frank Tassoni, JR., Ramsey, MN (US); Martin R. Willard, Burnsville, MN (US); Patrick A. Haverkost, Brooklyn Center, MN (US); Jonathan S. Stinson, Plymouth, MN (US); Peter G. Edelman, Maple Grove, MN (US); Jacob Drew Edick, Minneapolis, MN (US)

(73) Assignee: Boston Scientific Scimed Inc., Maple Grove, MN (US)

(21) Appl. No.: 14/691,107

(22) Filed: Apr. 20, 2015

Related U.S. Application Data
(60) Provisional application No. 61/982,546, filed on Apr. 22, 2014.

(51) Int. Cl. A61F 2/962

(52) U.S. Cl. CPC A61F 2/962; A61F 2002/9517

(57) ABSTRACT

A stent delivery catheter comprising at least one catheter shaft, the catheter shaft having an inner surface and an outer surface and a distal end and a proximal end, the catheter shaft defining a guidewire lumen, the guidewire lumen comprising a diameter defined by the inner surface of the catheter shaft, a stent disposed within the distal end of the catheter shaft and in contact with the inner surface of the catheter shaft, and a stylet disposed within the distal end of the catheter shaft, the stylet comprising a wave geometry.

### CROSS-REFERENCE TO RELATED APPLICATIONS

This application claims priority to U.S. Patent Provisional Application No. 61/982,546, filed Apr. 22, 2014, the entire contents of which is hereby incorporated herein by reference.

### BACKGROUND OF THE INVENTION

The present invention relates to delivery systems for self-expanding medical devices such as stents.

The present invention relates to catheter delivery devices for delivery of expandable medical devices to a treatment site in a patient's body lumen, and particularly to catheter delivery devices for self-expanding stents.

In a catheter assembly adapted for stent delivery, stylets or mandrels are typically preloaded into the lumen of the distal end of an inner catheter shaft, particularly for self-expanding stents, to maintain the patency of the inner shaft lumen, i.e. the guidewire lumen, during storage and handling. The self-expanding stent is crimped onto the outer surface of the inner catheter shaft and an outer catheter shaft or restraining tube is disposed over the stent. Stylets are typically straight with a circular cross-section.

Self-expanding stents continually exert an outward force to the interior surface of the outer catheter shaft or restraining tube which is commonly formed of from a polymer material, such as an elastomeric polymer material. Over time, this force can cause cold flow of the interior surface of the outer shaft allowing the stent to partially embed itself into the interior surface of the outer shaft. This can significantly increase the initial deployment force required to free the stent from the outer catheter shaft.

U.S. patent application Ser. No. 2013/0096482 filed Oct. 18, 2011 and published Apr. 18, 2013, discloses a stylet having a non-round cross-sectional shape adapted to reduce the adhesion or stickiness of contact between the stylet and the interior surface of the lumen of an elastomeric catheter through which it extends.

There remains a need in the art for a stylet that can mechanically disrupt the adhesive bond between the stent and the interior surface of the outer catheter shaft of restraining tube when removed prior to use in order to reduce the initial stent deployment force.

### SUMMARY OF THE INVENTION

In one aspect, the present invention relates to a stent delivery catheter comprising at least one catheter shaft, the catheter shaft having an inner surface and an outer surface and a distal end and a proximal end, the catheter shaft defining a guidewire lumen, the guidewire lumen comprising a diameter defined by the inner surface of the catheter shaft, a stent disposed within the distal end of the catheter shaft and in contact with the inner surface of the catheter shaft and a stylet disposed within the distal end of the catheter shaft, the stylet comprising a wave geometry.

In another aspect, the present invention relates to a stylet for insertion into the distal end of a catheter guide wire lumen, the stylet comprising a wave geometry.

In another aspect, the present invention relates to a method of maintaining the patency of a guidewire lumen of a stent delivery catheter, the catheter comprising at least one catheter shaft, the catheter having in inner surface and an outer surface, the inner surface having a distal end and a proximal end, the method comprising inserting a stylet in the distal end of the catheter shaft, the stylet comprising a wave geometry.

These and other aspects, embodiments and advantages of the present disclosure will become immediately apparent to those of ordinary skill in the art upon review of the Detailed Description and Claims to follow.

### BRIEF DESCRIPTION OF THE DRAWINGS

FIG. 1 is a side view of a rapid exchange stent delivery catheter system in accordance with an exemplary embodiment of the present disclosure, shown in the delivery state.

FIG. 2 is a side view of a distal portion of the rapid exchange stent delivery catheter system illustrated in FIG. 1, shown in the deployment state.

FIG. 3 is a partial longitudinal cross-section of a stent delivery system according to the invention.

FIG. 4 is a side view of a wavy stylet according to the invention.

FIG. 5 is a partial view of a stent delivery system similar to that shown in FIG. 3 having the wavy stylet disposed in the inner lumen of the catheter assembly.

### DETAILED DESCRIPTION OF THE INVENTION

While embodiments of the present disclosure may take many forms, there are described in detail herein specific embodiments of the present disclosure. This description is an exemplification of the principles of the present disclosure and is not intended to limit the disclosure to the particular embodiments illustrated.

The following detailed description should be read with reference to the drawings in which similar elements in different drawings are numbered the same. The drawings, which are not necessarily to scale, depict illustrative embodiments and are not intended to limit the scope of the invention. Those skilled in the art will recognize that the dimensions and materials discussed herein are merely exemplary and are not intended to limit the scope of the present invention.

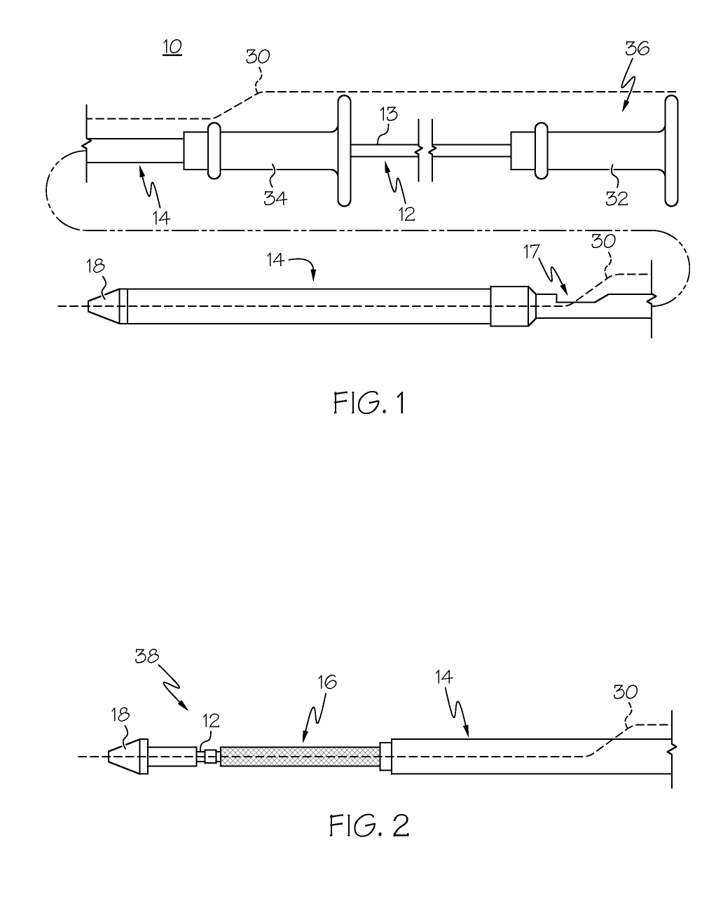

Turning now to the figures, FIG. 1 is a side view of one embodiment stent delivery catheter system 10 in a delivery state wherein the stent (not shown) is restrained by an outer member 14. Of course, this is only an exemplary embodiment and any other stent delivery catheter configuration may be employed herein without departing from the scope of the invention. Catheter 10 includes an inner member 12 that defines a guide wire lumen 13. Guide wire 30 is shown in phantom. Guide wire 30 is inserted in catheter at guide wire port 17 at the proximal end 36 of catheter 10. Catheter 10 is advanced over guide wire 30 to deliver and deploy a self-expanding stent 16, shown disposed on the distal end 38 of inner member 12 as shown in FIG. 2. Distal end 38 of catheter 10 includes a tapered or rounded distal tip 18 for ease of insertion.

FIG. 2 is a side view of a distal portion of stent delivery catheter 10 illustrated in FIG. 1, shown in the deployment state. Stent 16 may be pushed out of the outer member 14 via a pusher tube (not shown) or the outer member 14 can been pulled back to expose the distal end of the inner member 12 with stent 16 disposed thereon. These types of stent delivery catheters are disclosed in commonly assigned U.S. Pat. Nos. 8,444,685; 7,468,053; 7,115,109; 6,890,317; 6,723,071; and 6,592,549; the entire disclosures of which are all incorporated herein by reference.

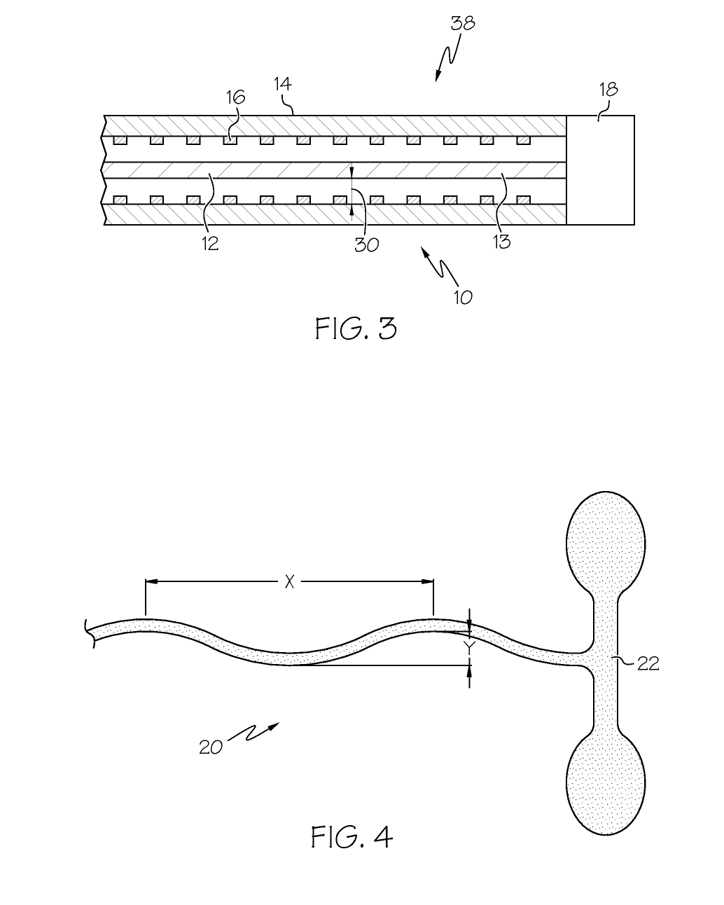

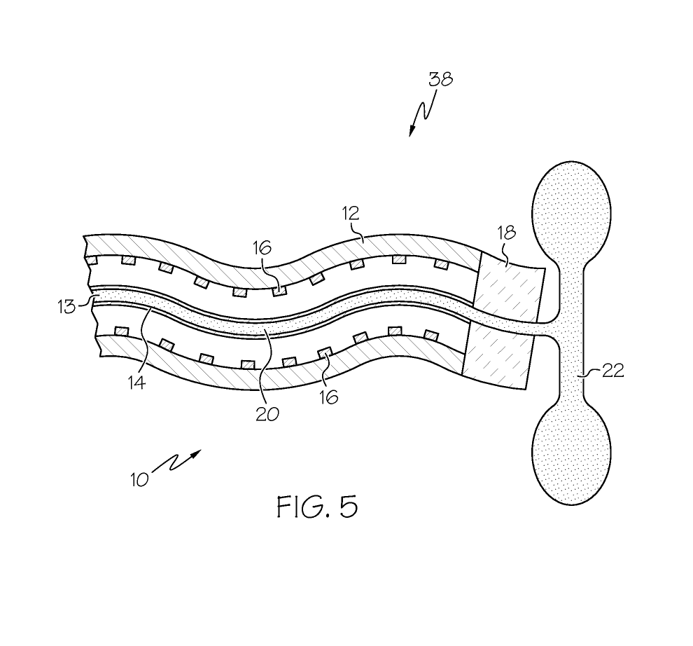

FIG. 3 is a longitudinal cross-section of the distal end 38 of the stent delivery catheter 10. Stent 16 is disposed on inner member 12 in a crimped state and restrained thereon by outer member 14. The inner surface of the inner member 12 defines the guide wire lumen 13. Guide wire 30 is shown in phantom therein.

The inner member 12 may be formed of any suitable biocompatible material which are known in the art such as a fluoropolymer, a block polyamide/polyether, high or low density polyethylene, silicone, or any other suitable material including a wide variety of polymers. The inner member may comprise a layered construction or may comprise a varying composition along the tube for pushability and trackability. The inner member can be formed of a combination of materials and may comprise a lubricious material on the inner surface of the inner member.

The inner and outer members may be formed from any suitable material or combinations thereof as is known in the art. Examples include, but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, other biocompatible polymers, other suitable materials, or mixtures, combinations, copolymers thereof, and the like.

This list is intended for illustrative purposes only, and not as a limitation on the scope of the present invention.

Inner and outer members may comprise different materials from the distal to the proximal end and may comprise layered composites as well.

The stylet disclosed herein for insertion into the distal end 38 of the inner member 12 suitably is formed such that it comprises features that provide a mechanical force to catheter assembly such that any bond that may have formed between stent 16 and the interior surface of the outer member 14 is caused to break.

FIG. 4 is a side view of one embodiment of a stylet 20 according to the invention having a wavy geometry. Stylet 20 may include a handle portion 22 for facilitating insertion of the stylet 20 into the guide wire lumen 13 and withdrawal therefrom such as by rotating the stylet 20 during insertion and withdrawal.

The handle portion 22 of the stylet 20 is also beneficial in that it can prevent the user from forgetting to remove it such as may be the case with a standard straight stylet prior to passing the stent delivery system through the introducer sheath or endoscope of a catheter assembly and into the patient. The size of the handle portion 22 suitably exceeds the entry diameter of the introducer and endoscope.

The wavy stylet 20 may take on any wave geometry or wave shaped configuration. The wave may have an amplitude y of about 2 mm to about 10 mm and a wavelength of about 2 mm to about 50 mm.

The stent delivery catheter of claim 1 wherein the stylet can have a one dimensional sine wave, a two dimensional sine wave or a helical three dimensional geometry.

It is important to note that the wave pattern or helical pattern of the wavy stylet need not comprise uniform waves in terms of wavelength and amplitube but rather, may also include patterns wherein these are varied. This applies to one, two and three dimensional geometries.

In some embodiments, the inner surface of the inner member 12 is defined by a first diameter and the stylet outer surface is defined by a second diameter, wherein the second diameter is larger than the first diameter.

In an alternative embodiment, a straight stylet as is well known in the art, is employed as usual during handling, packaging and storage of the stent delivery catheter 10 and removed at the time of use. The catheter can be packaged and shipped with a straight stylet in place to maintain dimensional stability which is then removed just prior to the time of use and replaced with a wavy stylet or mandrel.

In an alternative embodiment, the stylet is shaped from a single rod or wire with a non-circular cross-section such as in the shape of a star, an octagon, an oval, a square, a semi-circle, and so forth. The non-circular types of cross-sections may be fabricated in straight linear orientation along the length of the stylet or in a helix (“barber pole”) orientation along the length of the stylet. In addition to the overall geometry of the non-linear stylet, the irregular shaped cross-section would present asymmetrical pressure points upon the inner member inner diameter (ID) surface as the stylet is being withdrawn from the stent deliver system. The translating non-symmetrical pressure points would serve to cause micro-movement between the (outer diameter) OD of the inner member and the ID of the constrained stent.

In another embodiment, the stylet is fabricated from multiple wires in communication with each other in the form of a cable. Rather than having a smooth circular cross-section using a single round wire, the cable construction produces an irregular shaped cross-section. This shape imparts additional movement of inner member and stent as the stylet is withdrawn from the inner member lumen.

The cable may be composed of two, three, four, or more wires. All of the wires may have the same diameter or may have different diameters. The individual wires in the cable may have a diameter ranging from slightly smaller than the inner member lumen diameter to a dimater that is one-tenth of the diameter of the inner member lumen, for example. The diameter of a circle enveloping the transverse cross-section of the cable ranges from about 80% to about 98% of the diameter of the inner member lumen.

The helix of the wire strands in the cable range from about ⅓ to about 1/25 of the length of the stylet, not including the length of the handle on the distal end of the stylet.

The handle can be joined to an end of the cable by welding, by crimping of a coupler on the termination of the handle and the cable end, by joining the two components in an thermoset bond such as an epoxy bond, by encasing in a molten thermoplastic polymer that solidifies upon cooling, or by soldering or brazing, for exmaple.

Alternatively, the handle may be shaped from a portion of the cable so that the entire stylet is fabricated from one continuous piece of cable.

The cable can be formed of any of the suitable materials listed above, or may be composed of polymer filaments such as polyimide, polycarbonate, nylon, and polyethylene, or may be a mixture of metallic and polymeric filaments.

In an alternative embodiment, the proximal end of the cable may have a flat face perpendicular to the longitudinal axis of the stylet or may have a rounded or pointed end to facilitate easy insertion into the distal end of the catheter inner member lumen.

The stylet may be coated with a lubricious material such as polytetrafluoroethylene (PTFE) or silicone to facilitate insertion and removal of the stylet from the inner member lumen.

The wavy stylet 20 is inserted into the distal end 38 of the inner member 12 of the catheter 10. This cause gentle distortion of the inner member 12 which in turns puts stress on the stent 16 and the interior surface of the outer member 14 resulting mechanical force and disruption of any bond that may have occurred between the stent 16 and the interior surface of the outer member 14 resulting in lower initial deployment force.

The stylet may be formed from a variety of materials including, but not limited to metallic material and/or a metallic alloy, such as stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In some embodiments, stylet 20 is formed from 300 series stainless steel, 400 series stainless steel, 17-7 PH stainless steel, Co—Cr—Ni super alloy, Ni—Co—Cr—Mo super alloy and mixtures thereof.

The stylet 20 may also be formed of other sufficient rigid materials such as polymer materials having a sufficient surface lubricity to facilitate insertion and withdrawal of the stylet 20.

The catheter 10 is typically between 130 cm and 140 cm in length, the stylet 20 is typically between about 5 mm and 10 mm in length. In one embodiment, catheter 10 is 137 cm and stylet 20 is 7 mm.

A description of some embodiments of a stent delivery catheter, stylet for use therein and methods of using the same is contained in one or more of the following statements:

Statement 1. A stent delivery catheter, the stent delivery catheter comprising:

at least one catheter shaft, the catheter shaft having an inner surface and an outer surface and a distal end and a proximal end, the catheter shaft defining a guidewire lumen, the guidewire lumen comprising a diameter defined by the inner surface of the catheter shaft;

a stent disposed within the distal end of the catheter shaft and in contact with the inner surface of the catheter shaft; and

a stylet disposed within the distal end of the catheter shaft, the stylet comprising a wave geometry.

Statement 2. The stent delivery catheter of statement 1 wherein the wave comprises an amplitude y of about 2 mm to about 10 mm.

Statement 3. The stent delivery catheter of statement 1 wherein the stylet comprises a wavelength of about 2 mm to about 50 mm.

Statement 4. The stent delivery catheter of statement 1 wherein the stylet comprises a one dimensional sine wave.

Statement 5. The stent delivery catheter of statement 1 wherein the stylet comprises a two dimensional sine wave.

Statement 6. The stent delivery catheter of statement 1 wherein the stylet comprises a three dimensional helical geometry.

Statement 7. The stent delivery catheter of statement 1 wherein the stylet comprises a member selected from the group consisting of 300 series stainless steel, 400 series stainless steel, 17-7 PH stainless steel, Co-Cr-Ni super alloy, Ni-Co-Cr-Mo super alloy and mixtures thereof.

Statement 8. The stent delivery catheter of statement 1 wherein the catheter shaft inner surface is defined by a first diameter and the stylet outer surface is defined by a second diameter, wherein the second diameter is larger than the first diameter.

Statement 9. The stent delivery catheter of statement 1 wherein the stylet comprises a handle configured to rotate the stylus within the catheter lumen.

Statement 10. The stent delivery catheter of statement 1 wherein said at least one catheter shaft is an outer shaft, the stent delivery catheter further comprising a second shaft that is an inner shaft, the inner shaft having an inner surface and an outer surface, the inner surface of the catheter shaft defining the guidewire lumen, the inner catheter shaft having a distal end and a proximal end, the stylet is disposed within the distal end of the inner shaft.

Statement 11. The stent delivery catheter of statement 1 wherein the wave configuration comprises a series of peaks and valleys wherein the amplitude of the peaks and valleys is greater than the diameter of the guidewire lumen.

Statement 12. A stylet for insertion into the distal end of a catheter guide wire lumen, the stylet comprising a wave geometry.

Statement 13. The stylet of statement 12 wherein the wave is one, two or three dimensional.

Statement 14. The stylet of statement 12 comprising a member selected from the group consisting of a member selected from the group consisting of 300 series stainless steel, 400 series stainless steel, 17-7 PH stainless steel, Co—Cr—Ni super alloy, Ni—Co—Cr—Mo super alloy and mixtures thereof.

Statement 15. The stylet of statement 12 wherein the wave geometry comprises an amplitude y of about 2 mm to about 10 mm and a wavelength of about 2 mm to about 50 mm.

Statement 16. A method of maintaining the patency of a guidewire lumen of a stent delivery catheter, the catheter comprising at least one catheter shaft, the catheter having in inner surface and an outer surface, the inner surface having a distal end and a proximal end, the method comprising:

inserting a stylet in the distal end of the catheter shaft, the stylet comprising a wave geometry.

Statement 17. The method of statement 16 further comprising inserting a straight stylet into the catheter shaft prior to inserting the stylet comprising a wave geometry.

Statement 18. The method of claim 16 wherein the wave geometry is one, two or three dimensional.

Statement 19. The method of statement 16 wherein the stylet comprises a member selected from the group consisting of a member selected from the group consisting of 300 series stainless steel, 400 series stainless steel, 17-7 PH stainless steel, Co—Cr—Ni super alloy, Ni—Co—Cr—Mo super alloy and mixtures thereof.

Statement 20. The method of statement 16 wherein the wave geometry comprises an amplitude y of about 2 mm to about 10 mm and a wavelength of about 2 mm to about 50 mm.

Statement 21. A stent delivery catheter, the stent delivery catheter comprising:

at least one catheter shaft, the catheter shaft having an inner surface and an outer surface and a distal end and a proximal end, the catheter shaft defining a guidewire lumen, the guidewire lumen comprising a diameter defined by the inner surface of the catheter shaft;

a stent disposed within the distal end of the catheter shaft and in contact with the inner surface of the catheter shaft; and

a stylet disposed within the distal end of the catheter shaft, the stylet comprising a wave geometry.

Statement 22. The stent delivery catheter of statement 21 wherein the wave comprises an amplitude y of about 2 mm to about 10 mm.

Statement 23. The stent delivery catheter of statements 21 or 22 wherein the stylet comprises a wavelength of about 2 mm to about 50 mm.

Statement 24. The stent delivery catheter of any of statements 21-23 wherein the stylet comprises a one dimensional sine wave.

Statement 25. The stent delivery catheter of any of statements 21-23 wherein the stylet comprises a two dimensional sine wave.

Statement 26. The stent delivery catheter of any of statements 21-23 wherein the stylet comprises a three dimensional helical geometry.

Statement 27. The stent delivery catheter of any of statements 21-26 wherein the stylet comprises a member selected from the group consisting of 300 series stainless steel, 400 series stainless steel, 17-7 PH stainless steel, Co—Cr—Ni super alloy, Ni—Co—Cr—Mo super alloy and mixtures thereof.

Statement 28. The stent delivery catheter of any of statements 21-27 wherein the catheter shaft inner surface is defined by a first diameter and the stylet outer surface is defined by a second diameter, wherein the second diameter is larger than the first diameter.

Statement 29. The stent delivery catheter of any of statements 21-28 wherein the stylet comprises a handle configured to rotate the stylus within the catheter lumen.

Statement 30. The stent delivery catheter of any of statements 21-29 wherein said at least one catheter shaft is an outer shaft, the stent delivery catheter further comprising a second shaft that is an inner shaft, the inner shaft having an inner surface and an outer surface, the inner surface of the catheter shaft defining the guide wire lumen, the inner catheter shaft having a distal end and a proximal end, the stylet is disposed within the distal end of the inner shaft.

Statement 31. The stent delivery catheter of any of statements 21-30 wherein the wave configuration comprises a series of peaks and valleys wherein the amplitude of the peaks and valleys is greater than the diameter of the guide wire lumen.

Statement 32. The stent delivery catheter of any of statements 21-31 wherein the wave geometry comprises an amplitude y of about 2 mm to about 10 mm and a wavelength of about 2 mm to about 50 mm.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All published documents, including all US patent documents and US patent publications, mentioned anywhere in this application are hereby expressly incorporated herein by reference in their entirety. Any copending patent applications, mentioned anywhere in this application are also hereby expressly incorporated herein by reference in their entirety. Citation or discussion of a reference herein shall not be construed as an admission that such is prior art.

1. A stent delivery catheter, the stent delivery catheter comprising:
at least one catheter shaft, the catheter shaft having an inner surface and an outer surface and a distal end and a proximal end, the catheter shaft defining a guidewire lumen, the guidewire lumen defined by the inner surface of the catheter shaft;
a stent disposed within the distal end of the catheter shaft and in contact with the inner surface of the catheter shaft; and
a stylet disposed within the distal end of the catheter shaft, the stylet comprising a wave geometry.

2. The stent delivery catheter of claim 1 wherein the wave comprises an amplitude y of about 2 mm to about 10 mm.

3. The stent delivery catheter of claim 1 wherein the stylet comprises a wavelength of about 2 mm to about 50 mm.

4. The stent delivery catheter of claim 1 wherein the stylet comprises a one dimensional sine wave.

5. The stent delivery catheter of claim 1 wherein the stylet comprises a two dimensional sine wave.

6. The stent delivery catheter of claim 1 wherein the stylet comprises a three dimensional helical geometry.

7. The stent delivery catheter of claim 1 wherein the stylet comprises a member selected from the group consisting of 300 series stainless steel, 400 series stainless steel, 17-7 PH stainless steel, Co—Cr—Ni super alloy, Ni—Co—Cr—Mo super alloy and mixtures thereof.

8. The stent delivery catheter of claim 1 wherein the stylet having a distal end and a proximal end, the stylet outer surface is defined by a diameter, the diameter being larger at the distal end and tapering to the proximal end.

9. The stent delivery catheter of claim 1 wherein the stylet comprises a handle configured to rotate the stylus within the catheter lumen.

10. The stent delivery catheter of claim 1 wherein said at least one catheter shaft is an outer shaft, the stent delivery catheter further comprising a second shaft that is an inner shaft, the inner shaft having an inner surface and an outer surface, the inner surface of the catheter shaft defining the guidewire lumen, the inner catheter shaft having a distal end and a proximal end, the stylet is disposed within the distal end of the inner shaft.

11. The stent delivery catheter of claim 1 wherein the wave configuration comprises a series of peaks and valleys wherein the amplitude of the peaks and valleys is greater than the diameter of the guidewire lumen.

12. A stylet for insertion into the distal end of a catheter guide wire lumen, the stylet comprising a wave geometry.

13. The stylet of claim 12 wherein the wave is one, two or three dimensional.

14. The stylet of claim 12 comprising a member selected from the group consisting of a member selected from the group consisting of 300 series stainless steel, 400 series stainless steel, 17-7 PH stainless steel, Co—Cr—Ni super alloy, Ni—Co—Cr—Mo super alloy and mixtures thereof.

15. The stylet of claim 12 wherein the wave geometry comprises an amplitude y of about 2 mm to about 10 mm and a wavelength of about 2 mm to about 50 mm.

16. A method of maintaining the patency of a guidewire lumen of a stent delivery catheter, the catheter comprising at least one catheter shaft, the catheter having in inner surface and an outer surface, the inner surface having a distal end and a proximal end, the method comprising:
inserting a stylet in the distal end of the catheter shaft, the stylet comprising a wave geometry.

17. The method of claim 16 further comprising inserting a straight stylet into the catheter shaft prior to inserting the stylet comprising a wave geometry.

18. The method of claim 16 wherein the wave geometry is one, two or three dimensional.

19. The method of claim 16 wherein the stylet comprises a member selected from the group consisting of a member selected from the group consisting of 300 series stainless steel, 400 series stainless steel, 17-7 PH stainless steel, Co—Cr—Ni super alloy, Ni—Co—Cr—Mo super alloy and mixtures thereof.

20. The method of claim 16 wherein the wave geometry comprises an amplitude y of about 2 mm to about 10 mm and a wavelength of about 2 mm to about 50 mm.
